(19) United States
(12) Patent Application Publication
(10) Pub. No.: US 2026/0335019 A1
(43) Pub. Date: Oct. 29, 2026

(54) INTRAOPERATIVE ULTRASOUND COREGISTERED TO PREOPERATIVE IMAGING TECHNIQUE FOR AIDING NAVIGATION OF INSTRUMENTS DURING ENDOVASCULAR PROCEDURES

(71) Applicants: THE TRUSTEES OF DARTMOUTH COLLEGE, Hanover, NH (US); MARY HITCHCOCK MEMORIAL HOSPITAL, FOR ITSELF AND ON BEHALF OF DARTMOUTH-HITCHCOCK CLINIC, Lebanon, NH (US)

(72) Inventors: William Ross WARNER, Hanover, NH (US); Xiaoyao FAN, Hanover, NH (US); Richard POWELL, Lebanon, NH (US); Keith D. PAULSEN, Hanover, NH (US)

(21) Appl. No.: 19/107,393

(22) PCT Filed: Aug. 31, 2023
(86) PCT No.: PCT/US2023/031617

§ 371 Date: Feb. 27, 2025

Related U.S. Application Data
(60) Provisional application No. 63/402,610, filed on Aug. 31, 2022.

(51) Int. Cl. A61B 34/20; A61B 5/06; A61B 34/10; G06T 7/33

(52) U.S. Cl. CPC A61B 34/20; A61B 5/066; A61B 34/10; G06T 7/33; A61B 2034/102; A61B 2034/105; G06T 2207/10081; G06T 2207/10088; G06T 2207/10132; G06T 2207/30101

(57) ABSTRACT

A system for visualizing surgical tools in vessels includes CT or MRI for angiographic imaging; and an ultrasound machine with tracked probe and an image processor that receives the angiographic and ultrasound images. The image processor constructs a model of relevant vessels from the angiographic images; coregisters the ultrasound images to the model of relevant vessels, locates surgical tools in the ultrasound images, coregisters locations of the surgical tools to model of the relevant vessels; and displays images of the model of the relevant vessels with superimposed images of the surgical tools. A method includes modeling relevant vessels from preoperative images; coregistering the model of relevant vessels to tracked ultrasound images, localizing the relevant vessels and surgical tools in the relevant vessels; and coregistering the ultrasound images to the model of relevant vessels, then superimposing and displaying images of the surgical tools on the model of relevant vessels.

### PRIORITY

The present document claims priority to U.S. Provisional patent application 63/402,610 filed 31 Aug. 2022. The entire contents of the aforementioned patent application are incorporated herein by reference.

### BACKGROUND

Image guidance plays a significant role in surgical procedures.

Typically, medical images of the patient, such as MRI (magnetic resonance imaging) and CT (computed tomography), are acquired preoperatively and used for intra-operative guidance by providing 3-dimensional map surgeons can use to guide instrument placement. Additional images, such as fluoroscope images, three dimensional optical images, endoscope camera images, or other tool navigational data such as tool position data from tool position encoders, are then acquired intraoperatively so the tools can be positioned to a desired position in the tissue.

These techniques have become mainstream in neurosurgery procedures with growth into other surgical disciplines yet have not gained traction in other types of surgical disciplines. This is especially true in vascular and endovascular surgery where image-based surgical navigation for surgical guidance remains in its technological infancy.

Minimally invasive endovascular procedures have been on the rise. These typically involve plaque removals, angioplasties, or placement of vascular grafts, all done through very small incisions in arteries without requiring open exposures to the patient's anatomy. Performing surgeries with minimal invasiveness offers significant benefits to patients such as reduced pain, scarring, length of stay, and reduced surgical morbidity.

Many tools, such as guidewires and catheters, used for endovascular procedures are, at least in part, flexible; flexibility allows tools to follow blood vessels as they are inserted and navigated through the blood vessels to a portion of a patient's anatomy needing surgical intervention. For example, but not limitation, many endovascular procedures such as implantation of aortal endovascular implants for aneurysm repair, are done using tools and implants, including guidewires, inserted through an incision in a femoral artery. Tools, such as angioplasty catheters, inserted through the femoral artery are also used for procedures involving coronary arteries and may be used for procedures involving carotids and other arteries. Since these tools are flexible, tracking of tool portions located outside the patient is not adequate to precisely locate the distal, active, ends of those tools relative to patient anatomy. We note that electromagnetic tracking of special tools within the body has been demonstrated; this technique requires an electrical connection to a coil on the tool to provide current to electromagnetic coils utilized for localization. This technique requires creation of custom tools and suffers from inaccuracies when large amounts of metal are in the surgical field.

Many tools are formed of radio-opaque materials and locations of their distal, active, ends can be visualized by using X-ray fluoroscopy, repeated imaging with fluoroscopy is required to navigate those tools to a desired location within a patient's vasculature. However, when fluoroscopy is used to help navigate tools, these procedures come at the high cost of a radiation burden to both patient and vascular surgery staff as surgeons rely on repeated 2D angiograms and live video angiography for navigation of guidewires placement of grafts at locations planned from preoperative CT angiography. Further, repeated 2D angiograms also expose patients to repeated dosage of sometimes-toxic radio-opaque dyes that can have significant adverse effects on some patients; these dyes are particularly dangerous with patients having renal insufficiency. As the complexity of the patient anatomy or procedure increases, the radiation dose typically increases as well. Further, 2D angiograms do not give a full picture of the three-dimensional nature of sometimes tortuous arteries-while intraoperative CT can give a better representation of 3D arteries it comes with very high radiation dose.

There are several types of trackers that can be applied to a tool and provide both location and orientation information regarding that tool in an operating room environment. Most such tool-attached trackers require a unit herein referred to as a tracking receiver, although in some systems the tracking receiver transmits infrared or visible optical, or radio, signals to the trackers as well as receiving signals or reflected light from the trackers attached to the tools.

### SUMMARY

In an embodiment, a system for visualizing surgical tools and their relationship to structures requiring surgical intervention includes apparatus for generating angiographic images selected from a magnetic resonance imaging machine, an X-ray computed tomography machine, or an ultrasound machine; an ultrasound machine with an ultrasound probe; a tracker coupled to the ultrasound probe; a tracking receiver configured to locate the ultrasound probe; an image processor coupled to receive the angiographic images and to receive ultrasound images from the ultrasound machine. The image processor is configured to: construct a three-dimensional mesh model of relevant vessels from the angiographic images; locate surgical tools in the ultrasound images, coregister locations of the surgical tools to the angiographic images; and render and display images of the three-dimensional mesh model of the relevant vessels with superimposed images of the surgical tools.

In an alternative embodiment, a three-dimensional mesh model is derived by a processor from images obtained using a Doppler ultrasound machine with the processor configured to locate surgical tools in the ultrasound images, coregister locations of the surgical tools to the three-dimensional mesh model; and render and display images of the three-dimensional mesh model of the relevant vessels with superimposed images of the surgical tools.

In some embodiments, the three-dimensional model of the relevant vessels is deformable so it can be adjusted to conform to intraoperative ultrasound imaging. This allows the system to compensate for differences in posture, patient positioning, pressure exerted on the patient by ultrasound transducers, and pressure on arteries exerted through surgical tools between preoperative imaging and during surgery.

In another embodiment, a method for visualizing surgical tools and their relationship to structures requiring surgical intervention includes performing preoperative imaging to obtain preoperative images; constructing a three-dimensional mesh model of relevant vessels from the preoperative images; coregistering the three-dimensional mesh model of relevant vessels to an intrasurgical physical location, tracking an ultrasound transducer location in position and orientation while the ultrasound transducer obtains ultrasound images localizing surgical tools in the relevant vessels; coregistering the ultrasound images to the preoperative images; superimposing models of both the surgical tools and relevant vessels; and rendering and displaying images of the surgical tools and surrounding relevant vessels.

In another embodiment, a system for visualizing surgical tools and their relationship to body structures includes CT or MRI for generating angiographic images; and an ultrasound machine with a tracked probe and an image processor that receives the angiographic images and ultrasound images. The image processor constructs a model of relevant vessels from the angiographic images; coregisters the ultrasound images to the model of relevant vessels, locates surgical tools in the ultrasound images, and coregisters locations of the surgical tools to model of the relevant vessels; and displays images of the model of the relevant vessels with superimposed images of the surgical tools.

And in another embodiment, A method includes constructing a model of relevant vessels from preoperative images; coregistering the model of relevant vessels to tracked ultrasound images, localizing the relevant vessels and the surgical tools in the relevant vessels; and coregistering the ultrasound images to the model of relevant vessels, then superimposing and displaying images of the surgical tools on the model of relevant vessels.

### BRIEF DESCRIPTION OF THE DRAWINGS

FIG. 1 is a flowchart of a method of visualizing tool placement in arteries of a patient to assist a surgeon in placing the tools in desired relationships with anatomic structures of the patient.

FIG. 2 is a block diagram of a system configured for the method of FIG. 1.

FIG. 3A is a doppler ultrasound image showing artery and vein, illustrating differences in fluid flow between arteries and veins.

FIG. 4A and FIG. 4B are images of a stent, 4A being of the stent outside a body showing what they look like, and 4B as imaged by ultrasound in a body.

FIG. 4C illustrates the stent of FIG. 4B after segmentation.

FIG. 5A and FIG. 5B are images of a guidewire 5A being of the guidewire outside a body showing what they look like, and 5B as imaged by ultrasound in a body.

FIG. 5C illustrates the guidewire of FIG. 5B after segmentation.

FIG. 6 illustrates a stent in a phantom as used to verify functionality ex-vivo.

### DETAILED DESCRIPTION OF THE EMBODIMENTS

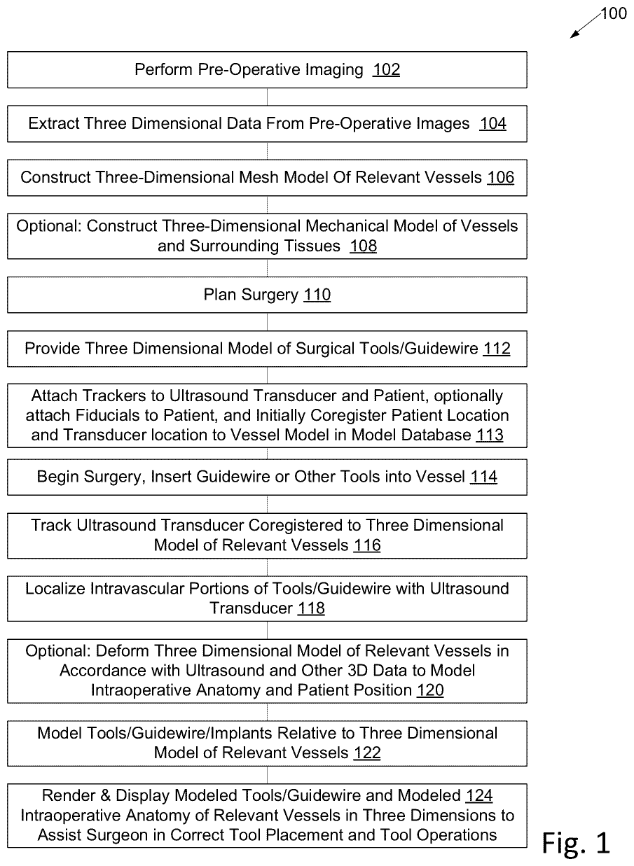

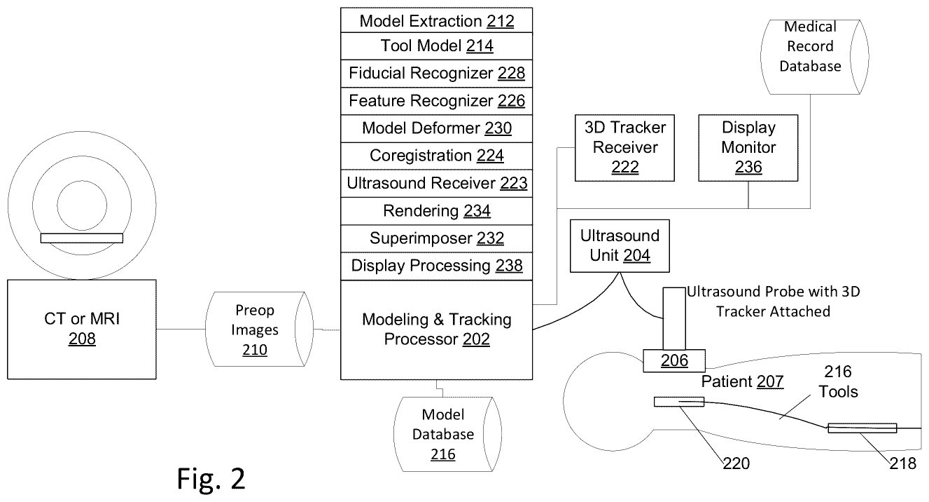

We propose a method 100 (FIG. 1) of visualizing intravascular surgical tool placement operable with a system 200 (FIG. 2) that includes an image processor 202, and ultrasound system 204 with ultrasound transducer 206; in alternative embodiments the ultrasound image-processing system is located within the transducer and coupled to display images by wire or short-range radio (such as Bluetooth) on a display device such as a smartphone or tablet computer.

The method 100 begins with performing 102 pre-operative imaging using an X-ray computed tomography (CT) machine or a magnetic resistance imaging (MRI) machine 208 to obtain a database of preoperative images 210; typically, a contrast medium such as an iodine or gadolinium dye is used during this imaging to perform CT-angiography or MR-angiography where blood vessels are highlighted, although surrounding tissues are also visible in the CT or MRI-generated preoperative 3D angiographic images. A 3D model of the arteries of interest is constructed from these 3D angiographic images. In an alternative embodiment, preoperative imaging is performed with ultrasound, using a tracker on the ultrasound probe, so that a preoperative 3D model can be built of the arteries of interest from images obtained by the tracked ultrasound probe. In another alternative embodiment, preoperative imaging is performed with Doppler ultrasound, using a tracker on the ultrasound probe, so that a preoperative 3D model can be built of the arteries of interest from images obtained by the tracked ultrasound probe; Doppler ultrasound highlights blood flowing in arteries and aids identification of arteries in ultrasound images but extracting artery information from ultrasound image stack and identifying surgical tools and implants from the ultrasound images can be done with or without Doppler.

Since Doppler highlights blood flow, the ability to extract artery information without Doppler may be of particular use in performing revascularization surgeries where blood flow is impaired.

In an alternative embodiment, a three-dimensional mesh model is derived by a processor from images obtained either preoperatively or as early-intraoperative images as surgery begins using a Doppler ultrasound machine with the processor configured to locate surgical tools in the ultrasound images, coregister locations of the surgical tools to the three-dimensional mesh model; and render and display images of the three-dimensional mesh model of the relevant vessels with superimposed images of the surgical tools.

In some embodiments, fiducials are attached to the patient prior to performing 102 the pre-operative imaging in order to assist coregistration. In an alternative embodiment, preoperative images are generated from multiple ultrasound images that may be acquired for surgical planning or acquired immediately before surgery or as surgery begins, the ultrasound images generated with an ultrasound probe tracked in three dimensions and structures tracked from image to adjacent image—when using ultrasound, power doppler techniques can map arterial blood flow without need for injected contrast media. Next, three-dimensional data is extracted 104 and a three-dimensional mesh model of relevant vessels is constructed 106 from the preoperative images 210 by executing model extraction code 212 on image processor 202; in embodiments this is done by first segmenting each two dimensional ultrasound image in a stack of images made with a tracked scanner, identifying corresponding structures in each image, and constructing a 3 dimensional model therefrom. In an alternative embodiment, a three-dimensional ultrasound scan is used instead of successive two dimensional scans so that identifying corresponding structures in each image is not necessary. In some embodiments, the three-dimensional mesh model of relevant vessels extracted from a stack of two dimensional ultrasound images or from a three dimensional ultrasound image includes a three-dimensional mechanical model 108 of both the relevant vessels and surrounding tissues, including bony tissues. The three-dimensional mesh model of the relevant vessels is stored in model database 212.

These constructed models based on extracted structures from the database of preoperative images 210 may be used with raw images from the database of preoperative images used to plan 110 a surgical intervention to repair aneurisms or perform other necessary surgical tasks.

A three-dimensional model of each tool to be used during the planned surgery, such as but not limited to endoscopes, implants, catheters, and guidewires, is also provided 112 in some embodiments and is also stored in model database 212. In an alternative embodiment a three-dimensional model of the tool is generated from ultrasound scans including the tool. This three-dimensional model in some embodiments, such as those used with flexible, deployable, arterial grafts for treating aneurysms, incorporates a mechanical model of implants or guidewires allowing for bending and, in many embodiments the mechanical model of implants or guidewires is also deformable to allow for bending of guidewires, deformation of soft implants, and similar factors. When deformable, the mechanical model may include portions that are indicated as bendable, maximum bend radius for bends, and portions indicated as not bendable.

Once the surgical intervention is planned 110, fiducials and/or trackers may be attached 113 to the patient and the ultrasound transducer 206 to aid coregistration of the three-dimensional mesh model of relevant vessels to the patient's intrasurgical physical location. The locations of patient, the location of the ultrasound transducer, and the three-dimensional mesh model of the relevant vessels are coregistered. An initial sweep of the ultrasound transducer over the region of interest of the patient with the relevant vessels may be performed so that an initial coregistration can be performed. Once this initial coregistration is performed, the coregistration is maintained, or in an alternative embodiment re-registered to the three-dimensional model of the artery, with each successive sweep of the ultrasound transducer over the region of interest as the patient moves or is moved and/or tools are inserted into vessels during surgery, since either patient movement or tool insertion can move or bend the artery.

Coregistration is performed by processor 202 executing coregistration code 224. Maintenance of coregistration throughout surgery is, in some embodiments, assisted by recognizing fiducial locations and/or tracker locations by fiducial recognition routines 228 executing on processor 202. In alternative embodiments, maintenance of coregistration throughout surgery is assisted by recognition of landmarks such as bodily structures that are easily recognized in images and provide high contrast in ultrasound images, such as bone, the artery of interest, or other visible structures. For purposes of this document, the term “recognizable landmark” includes both fiducials and bodily structures easily recognizable in images.

Surgery then begins 114 and the required tools 216, which may include guidewires, endoscopes, staplers, endovascular grafts, and other tools, are inserted into the relevant vessels. In an embodiment, the tools are inserted through the femoral artery (not shown) to reach the aorta 218 as the relevant vessel. In another embodiment, tools 216 are inserted through the femoral artery (not shown) and aorta 218 to reach the carotid artery 220.

In alternative embodiments, other arteries may be of interest as requiring surgical attention, such as but not limited to renal arteries, Iliac arteries, femoral arteries, subclavian arteries, brachial arteries, radial arteries, and coronary arteries. In these embodiments, tools 216 are inserted through an incision in an appropriate, accessible, artery and threaded through arteries to reach the artery of interest. In embodiments addressing lesions of pulmonary arteries, tools 216 are inserted through an incision in an appropriate, accessible vein and threaded through veins and the right side of the heart and to reach the artery of interest. Similarly, when accessing atria of the heart veinous access may be used. For other arteries, such as the carotids and certain arteries of the limbs, access may be achieved through an incision in the same artery that is to be operated on but at a different location in the artery from the site in the artery where surgery is to be performed; thus it is possible to perform surgery upon two or more locations in the carotid or femoral arteries through a single incision into the artery of interest.

The ultrasound transducer 206 location is tracked 116 both in position and orientation by tracking receivers 222 while ultrasound transducer 206 is used both to localize the relevant vessels and to localize 118 tools 216 in the relevant vessels. Ultrasound images are brought into processor 202 through ultrasound receiver routines 223, features are extracted with vessels and tools recognized, and are coregistered to the preoperative images.

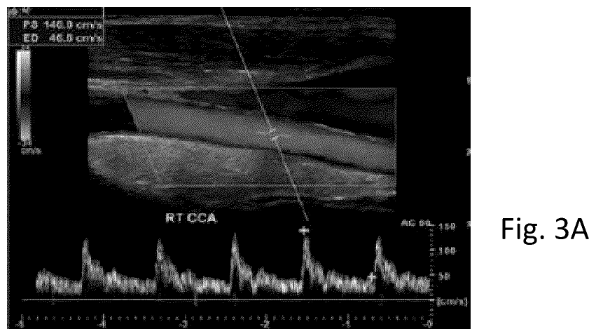

During extraction of the three dimensional data 104 from the preoperative images, creation of the three-dimensional model 106, and extraction of features with recognition of vessels and tools recognized in the ultrasound images, locations and orientations of the vessels and of these tools 216 are recognized by feature recognition routines 226; once identified in the images, locations of the tools in three dimensions are computed based upon position and orientation of the ultrasound transducer 206. Feature recognition routines 226, also known as a feature recognizer, in some embodiments has two primary sections, a segmenter that processes raw images to identify shapes as regions in the images while providing segmented images, and a shape recognizer that recognizes these shapes and shapes in adjacent “slice” images to identify blood vessels, bones, tools, and other structures. In some embodiments, additional features of patient anatomy, such as bony parts of surrounding tissues recognizable as hard echoes in ultrasound and relevant vessels recognizable through “power doppler” or similar observations of doppler shift induced by blood movement in those vessels as illustrated in FIG. 3a, are also both recognized by feature recognition routines and used to help constrain the three-dimensional model of relevant vessels during deformation 120 and to aid coregistration of the ultrasound images to the three-dimensional model of relevant vessels. A stack of 2-D ultrasound images can be used while recognizing similar shapes in adjacent images of the stack to recognize an artery from which a three-dimensional model of the artery may be generated; in an alternative embodiment the three-dimensional model of the artery may be extracted from a three-dimensional ultrasound scan. In some embodiments, feature recognition routines 226 include one or more of a KNN classifier and neural network classifier as known in the art of machine-learning-based classifiers. In some embodiments, a region growing method is used in feature recognition routines to identify entire artery and tool images in the ultrasound images. In a particular embodiment, power-doppler images of relevant vessels from current ultrasound are coregistered to, and then displayed as colored overlays on preoperative images. In an alternative embodiment, another colored overlay is generated highlighting other anatomic structures, such as but not limited to bone, recognized by feature recognizer 226 and used to help coregister ultrasound images to each other and to preoperative images.

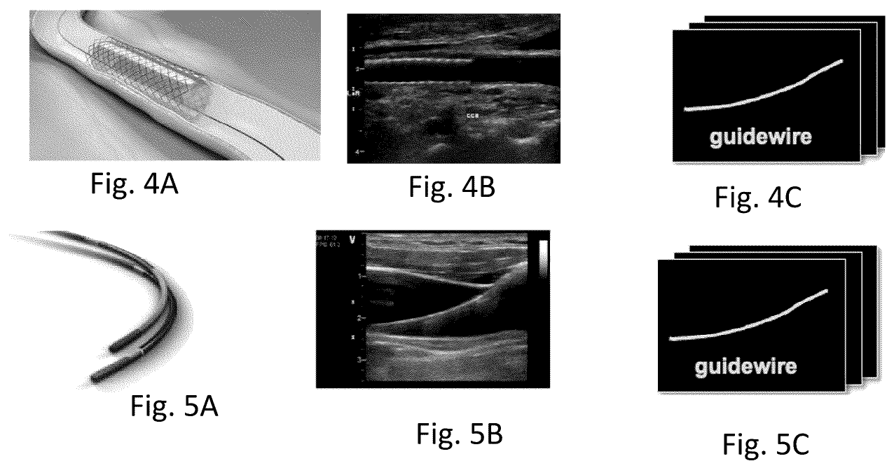

When using two dimensional ultrasound imaging devices, a series of images is obtained, together with tracked ultrasound transducer position and angle for each image, and both relevant vessels and tools are traced through the series of ultrasound images so a three-dimensional ultrasound image of the relevant vessels and tools can be constructed in memory. Example ultrasound images of tools are illustrated in FIG. 4B and FIG. 5B, with FIG. 4A and FIG. 5A illustrating the same tools ex vivo. When using three-dimensional ultrasound, with the aid of tracked locations of the ultrasound transducers it is possible to use tracker information to aid splicing three-dimensional ultrasound images together to provide a larger three dimensional ultrasound image in memory that covers the entire region of interest. The artery model can also be extracted from this three-dimensional ultrasound image.

Relevant vessels, such as but not limited to the aorta 218, carotid arteries 220, and peripheral arteries such as but not limited to the femoral and brachial arteries, are soft tissues that may deform both with patient movements and tool 216 presence within them. In some embodiments, the three-dimensional mesh model of the relevant arteries includes a model of nearby tissues, that is deformed 120 by execution of model deformer 230 routines; in embodiments this deformation is constrained to match current locations of fiducials as determined by fiducial recognizer 228 and/or vessels as extracted by feature recognizer 226. This deformation modeling is provided in part because pressure on patient 207 exerted by operators through ultrasound transducer 206, presence of tools used, or changes in bodily position, may displace soft tissues during the surgical intervention. Since some tools, including guidewires, and many stents and catheters, are also flexible and may bend while being inserted into the arteries, deformation modeling is also performed on the tool models; the tool models may incorporate rigid and flexible sections of the tool model to model tools with rigid and flexible sections. In some embodiments, intraoperative power-doppler ultrasound images, such as sometimes used to monitor flow in vessels like arteries, are coregistered to, and displayed as, colored overlays on tomographic slice images derived from the mechanical model of relevant vessels and nearby tissues as deformed to correspond to tracked and coregistered fiducials and landmarks to assist a surgeon in determining intraoperative locations and shapes of these tissues.

At this point, deformed and localized models of both relevant vessels and tools are available. These are superimposed by superimposer 232 routines, and both the surgical tools and relevant vessel models are rendered by rendering routines 234 to provide images of the surgical tools and surrounding relevant vessels that are displayed on display 236 by display routines 238 executed on processor 202. The displayed images illustrating the surgical tools and the surrounding relevant vessels or other structures requiring surgical intervention are expected to be useful to a surgeon throughout surgery to best guide the tools into desirable locations and orientations as they help him observe what he's doing while performing the surgical intervention. These displayed images are expected to be particularly useful while performing minimally invasive endovascular surgery using tools threaded through small incisions in arteries remote from the vessels requiring surgical intervention.

Since surgery is often a lengthy process, the steps 118-124 of localizing 118 tools, recognizing features and fiducial locations, model deformation, model execution and rendering, and display are repeated as necessary throughout surgery to allow the surgeon to guide the tools into desirable locations and observe the surgical intervention as it is performed. During insertions of tools and performing surgery the tools typically require rotation, twisting through steering wires, withdrawal/insertion, and other manipulations as they are pass through vessels and are steered to the relevant vessels.

In particular embodiments, in addition to modeling relevant vessels, nearby critical structures such as renal arteries and neural structures are also modeled so that they may be rendered and highlighted in rendered images—this is expected to help avoid undesirable outcomes such as damaged neural structures or obstructed blood flow to kidneys. In these embodiments, intraoperative power-doppler images may be coregistered to, and displayed as superimposed on, tomographic slices rendered from the mechanical model of relevant vessels and nearby critical structures as deformed to align fiducials and landmarks extracted from intraoperative ultrasound images. These power-doppler images superimposed on model images may provide surgeons with valuable information regarding potential damage to nearby critical structures.

During 3-D extraction 104, in embodiments, recognition of structures may be manual, semi-automatic where an operator identifies a shape in one tomographic slice and boundaries of that shape are automatically recognized in the remainder of that shape and in adjacent tomographic slices, fully automatic based on such factors as the high radio density of bone, guidewires, some implants, and tools, and combinations thereof.

During deformation 120, the mechanical model in some embodiments is constrained with differences in deformability between tissue types such as hard tissues like bone being modeled as less deformable than soft tissues like fat, muscles, and intestines.

The system and method herein described leverage techniques of surgical navigation and modelling, some of which have been used in neurosurgery, and apply them to vascular surgery to reduce the radiation dose to patients and surgical staff intraoperatively.

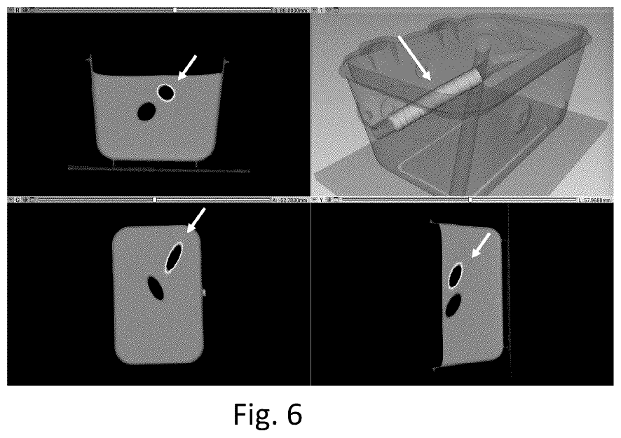

The apparatus and method herein described can track guidewires and view stents as placed into arteries. This has been demonstrated in a phantom (FIG. 6) having fluid-filled 8 mm-diameter holes representing arteries with guidewires or stents placed in the holes. The white arrow in each quadrant indicates the stent. The top right quadrant shows the reconstructed stent model diagonally in the phantom, the phantom represented as a tub. In this demonstration, two-dimensional ultrasound images were obtained using a 30-second sweep over the phantom with trackers monitoring angle and position of the ultrasound transducer, then smoothing the images with a circular Hough transform. The images were segmented into shapes, and shapes recognized, a 3-D model of the phantom showing the holes and guidewires or stents was constructed in 30 seconds to permit imaging at a rate useful during surgery.

The present system and method are useful for endovascular surgeries on the carotids, as well as for endovascular surgeries on the aorta, femoral, brachial, and other limb arteries as well as other systemic arteries like the renal arteries. We also expect the system to be useful for endovascular surgeries on other blood vessels such as major arteries feeding limbs and for treatment of stroke by endovascular surgical reopening of arteries or stenting of leaking arteries. In time, as scan and processing times are reduced with improved and parallel processing hardware, we expect that images could be obtained and processed rapidly enough to permit use of the technique on coronary arteries. In some embodiments it is possible to use transesophageal ultrasound with a probe inserted through the mouth, or ultrasound using an ultrasound probe inserted through another bodily orifice, to observe and provide guidance for surgeries on arteries such as the coronary arteries to enhance ultrasound visibility that may be degraded by such structures as the lungs.

We note that using CT angiography or MR angiography as preoperative imaging permits the system to portray bifurcations and branches of arteries that cannot be seen easily in the intraoperative ultrasound images as well as those which can be seen in the ultrasound images.

### Combinations

The various features herein described may be combined in various ways. Among the ways these features can be combined as anticipated by the inventors are:

A system designated A for visualizing surgical tools and their relationship to structures requiring surgical intervention includes apparatus for generating angiographic images selected from the group consisting of a magnetic resonance imaging machine, an X-ray computed tomography machine, or an ultrasound machine which may or may not be a Doppler ultrasound machine; and an ultrasound machine with an ultrasound probe with a tracker coupled to the ultrasound probe. A tracking receiver is configured to locate the ultrasound probe and provide locations and orientations of the ultrasound probe to an image processor also coupled to receive the angiographic images and to receive ultrasound images from the ultrasound machine. The image processor is configured to: construct a three-dimensional mesh model of relevant vessels from the angiographic images; coregister the ultrasound images or a three-dimensional mesh model extracted from the ultrasound images to the three-dimensional mesh model of relevant vessels, locate surgical tools in the ultrasound images, and coregister locations of the surgical tools to the three-dimensional mesh model of the relevant vessels; and render and display images of the three-dimensional mesh model of the relevant vessels with superimposed images of the surgical tools.

A system designated AA including the system designated A wherein the image processor is further configured to identify corresponding anatomic features in the angiographic images and the ultrasound images, and to use these corresponding anatomic features while coregistering the ultrasound images or a three-dimensional mesh model extracted from the ultrasound images to the three-dimensional mesh model of relevant vessels.

A system designated AB including the system designated A or AA further comprising computer models of the surgical tools.

A system designated AC including the system designated AB wherein the superimposed images of the surgical tools are rendered from the computer models of the surgical tools.

A system designated AD including the system designated AB or AC wherein the computer models of the tools are deformable.

A system designated AE including the system designated A, AA, AB, AC, or AD wherein the three-dimensional mesh model of relevant vessels is deformable, and where the three-dimensional mesh model of relevant vessels is deformed during coregistering of the ultrasound images, or a three-dimensional mesh model extracted from the ultrasound images to the three-dimensional mesh model of relevant vessels.

A system designated AF including the system designated A, AA, AB, AC, AD, or AE wherein reference points of bony tissues are recognized in the angiographic images and included in the three-dimensional mesh model of the relevant vessels, reference points of bony tissues are identified in the ultrasound images, and these reference points of bony tissues in the ultrasound images are used to aid coregistering of the ultrasound images to the three-dimensional mesh model of relevant vessels.

A system designated AG including the system designated A, AA, AB, AC, AD, AE, or AF wherein fiducials are used to aid coregistering of the ultrasound images to the three-dimensional mesh model.

The system designated AH including the system designated A, AA, AB, AC, AD, AE, AF, or AG where the apparatus for generating angiographic images is a Doppler ultrasound machine.

A method designated B for visualizing surgical tools and their relationship to structures requiring surgical intervention includes performing pre-operative imaging to obtain a database of preoperative images; constructing a three-dimensional mesh model of relevant vessels from the preoperative images; coregistering the three-dimensional mesh model of relevant vessels to an intrasurgical physical location, tracking an ultrasound transducer location in position and orientation while the ultrasound transducer obtains ultrasound images localizing the relevant vessels and the surgical tools in the relevant vessels; and coregistering the ultrasound images to the three-dimensional mesh model of relevant vessels, while deforming the three-dimensional mesh model of relevant vessels to conform to the ultrasound images localizing the relevant vessels. The method also includes superimposing models of the surgical tools on the three-dimensional mesh model of relevant vessels; and rendering and displaying images of the surgical tools and surrounding relevant vessels.

A method designated BA including the method designated B wherein the pre-operative imaging is performed with computed X-ray tomography (CT) or magnetic resonance imaging (MRI).

A method designated BB including the method designated B or BA further includes extracting a three-dimensional model from the ultrasound images and where coregistering the ultrasound images to the three dimensional mesh model of relevant vessels includes coregistering the three-dimensional from the ultrasound images to the three dimensional model of relevant vessels.

A method designated BC including the method designated BA, BB, or B wherein the image processor is further configured to identify corresponding anatomic features in the angiographic images and the ultrasound images, and to use these corresponding anatomic features while coregistering the ultrasound images to the three-dimensional mesh model of relevant vessels.

A method designated BD including the method designated BC, BB, BA, or B further comprising deforming the computer models of the tools to conform with the ultrasound images.

A method designated BE including the method designated BD, BC, BB, BA, or B wherein the three-dimensional mesh model of relevant vessels is deformed during coregistering of the ultrasound images, or a three-dimensional mesh model extracted from the ultrasound images to the three-dimensional mesh model of relevant vessels.

A method designated BF including the method designated BE, BD, BC, BB, BA, or B wherein reference points of bony tissues are recognized in the preoperative images and included in the three-dimensional mesh model of the relevant vessels, reference points of bony tissues are identified in the ultrasound images, and these reference points of bony tissues in the ultrasound images are used to aid coregistering of the ultrasound images to the three-dimensional mesh model of relevant vessels.

A method designated BF including the method designated BF, BD, BD, BB, or B where fiducials are used to aid coregistering of the ultrasound images to the three-dimensional mesh model.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

1. A system for visualizing surgical tools and their relationship to structures requiring surgical intervention comprising:
an ultrasound machine with an ultrasound probe;
apparatus for generating images highlighting arteries selected from the group consisting of: a magnetic resonance imaging machine, an X-ray computed tomography machine, and the ultrasound machine;
a tracker coupled to the ultrasound probe;
a tracking receiver configured to locate the ultrasound probe;
an image processor coupled to receive the images highlighting arteries and to receive ultrasound images from the ultrasound machine, the image processor configured to:
construct a three-dimensional mesh model of relevant vessels from the images highlighting arteries;
coregister the ultrasound images or a three-dimensional mesh model extracted from the ultrasound images to the three-dimensional mesh model of relevant vessels, locate surgical tools in the ultrasound images, and coregister locations of the surgical tools to the three-dimensional mesh model of the relevant vessels; and
render and display images of the three-dimensional mesh model of the relevant vessels with superimposed images of the surgical tools.

2. The system of claim 1 wherein the image processor is further configured to identify corresponding anatomic features in the images highlighting arteries and the ultrasound images, and to use these corresponding anatomic features while coregistering the ultrasound images or the three-dimensional mesh model extracted from the ultrasound images to the three-dimensional mesh model of relevant vessels.

3. The system of claim 1 further comprising computer models of the surgical tools, and where the superimposed images of the surgical tools are rendered from the computer models of the surgical tools.

4. The system of claim 3 wherein the computer models of the surgical tools are deformable.

5. The system of claim 1 wherein the three-dimensional mesh model of relevant vessels is deformable, and where the three-dimensional mesh model of relevant vessels is deformed during coregistering of the ultrasound images or the three-dimensional mesh model extracted from the ultrasound images, to the three-dimensional mesh model of relevant vessels.

6. The system of claim 1 wherein the processor is configured to periodically update coregistration of the ultrasound images to the three-dimensional mesh model of relevant vessels.

7. The system of claim 1 wherein reference points of bony tissues are recognized in the images highlighting arteries and included in the three-dimensional mesh model of the relevant vessels, reference points of bony tissues are identified in the ultrasound images, and these reference points of bony tissues in the ultrasound images are used to aid coregistering of the ultrasound images to the three-dimensional mesh model of relevant vessels.

8. The system of claim 1 wherein the ultrasound machine is a doppler ultrasound machine.

9. The system of claim 1, wherein fiducials are used to aid coregistering of the ultrasound images to the three-dimensional mesh model.

10. A method for visualizing surgical tools and their relationship to structures requiring surgical intervention comprising:
performing preoperative or early intraoperative imaging to obtain a database of preoperative images;
constructing a three-dimensional mesh model of relevant vessels from the preoperative images;
coregistering the three-dimensional mesh model of relevant vessels to an intrasurgical physical location,
tracking an ultrasound transducer location in position and orientation while the ultrasound transducer obtains ultrasound images localizing the relevant vessels and the surgical tools in the relevant vessels;
coregistering the ultrasound images to the three-dimensional mesh model of relevant vessels, while deforming the three-dimensional mesh model of relevant vessels to conform to the ultrasound images localizing the relevant vessels;
superimposing models of the surgical tools on the three-dimensional mesh model of relevant vessels; and
rendering and displaying images of the surgical tools and surrounding relevant vessels.

11. The method of claim 10 wherein the preoperative or early intraoperative imaging is performed with computed X-ray tomography (CT) or magnetic resonance imaging (MRI).

12. The method of claim 10 wherein the preoperative or early intraoperative images are obtained as surgery begins.

13. The method of claim 10 where the preoperative or early intraoperative images are ultrasound images.

14. The method of claim 10 further comprising extracting a three-dimensional model from the ultrasound images and where coregistering the ultrasound images to the three-dimensional mesh model of relevant vessels comprises coregistering the three-dimensional from the ultrasound images to the three-dimensional mesh model of relevant vessels.

15. The method of claim 10 further comprising to identifying corresponding anatomic features in the preoperative images and the ultrasound images, and to use these corresponding anatomic features while coregistering the ultrasound images to the three-dimensional mesh model of relevant vessels.

16. The method of claim 10 further comprising deforming computer models of the tools to conform with the ultrasound images.

17. The method of claim 10 wherein the three-dimensional mesh model of relevant vessels is deformed during coregistering of the ultrasound images or a three-dimensional mesh model extracted from the ultrasound images to the three-dimensional mesh model of relevant vessels.

18. The method of claim 10 wherein reference points of bony or other tissues are recognized in the preoperative images and included in the three-dimensional mesh model of the relevant vessels, reference points of bony tissues are identified in the ultrasound images, and these reference points of bony tissues in the ultrasound images are used to aid coregistering of the ultrasound images to the three-dimensional mesh model of relevant vessels.

19. The method of claim 10, wherein fiducials are used to aid the coregistering of the ultrasound images to the three-dimensional mesh model.
